(19) United States
(12) Patent Application Publication
(10) Pub. No.: US 2016/0199531 A1
(43) Pub. Date: Jul. 14, 2016

(54) PACKAGED PROBIOTIC COMPOSITION AND USES THEREOF

(71) Applicant: Micuri ApS, Hillerod (DK)

(72) Inventors: Brian Nielsen, Gorlose (DK); Eric Nielsen, Frederiksvaerk (DK); Helene Kähler Hjenner, Hillerod (DK)

(21) Appl. No.: 15/080,288

(22) Filed: Mar. 24, 2016

Related U.S. Application Data
(63) Continuation of application No. 14/359,224, filed on May 19, 2014.

(30) Foreign Application Priority Data
Nov. 25, 2011 (EP) 11190803.4

(51) Int. Cl. A61L 15/36; A61L 26/00; A61L 15/42; A61L 15/44; A61L 15/24

(52) U.S. Cl. CPC A61L 15/36; A61L 15/44; A61L 15/24; A61L 15/425; A61L 26/008; A61L 26/0066; A61L 26/0014; A61L 26/0023; A61K 2035/115

(57) ABSTRACT

The present invention relates to a packaged probiotic composition which is useful for treating or preventing bacterial colonisation in wounds and tissue. A characteristic feature of the invention is that the probiotic composition contains a probiotic microorganism which is in direct contact with water during storage of the probiotic composition. The invention furthermore pertains to methods of using and producing the probiotic composition.

### FIELD OF THE INVENTION

The present invention relates to a packaged probiotic composition which is useful for treating or preventing bacterial colonisation in wounds and tissue. A characteristic feature of the invention is that the probiotic composition contains a probiotic microorganism which is in direct contact with water during storage of the probiotic composition. The invention furthermore pertains to methods of using and producing the probiotic composition.

### BACKGROUND OF THE INVENTION

It has been known for decades that probiotic bacteria improve the health of the digestive system, also if inflammation or wounds occur in the bowel (treating Colitis Ulcerosa or Morbus Crohn).

Some prior art documents furthermore describe probiotic compositions for topical applications.

WO 2008/074,331 discloses the use of lyophilized lactic acid bacteria in wound dressings. Such wound dressings may also contain absorbent materials for absorbing exudates from the wound. WO 2008/074,331 also discloses that such wound dressings may contain water, but it does not describe that lactic acid bacteria are in contact with water during storage.

WO 00/61,201 discloses a method of inhibiting microbial infection which is often observed in connection with the use of diapers and other sanitary products. More specifically, WO 00/61,201 describes that an aqueous suspension of lactic acid-producing bacteria is applied to the sanitary product, the product is dried and the sanitary product is subsequently used.

However, it does not describe that lactic acid-producing bacteria of the sanitary product are in contact with water during storage of the sanitary product.

### SUMMARY OF THE INVENTION

The present inventor has realised that the above-mentioned approaches of the prior art suffer from significant draw-backs.

The use of dried probiotic microorganisms reported in the prior art results in a substantial loss of viability of the microorganisms. Yet a disadvantage of this approach is that it takes time for the viable microorganisms to recover when resuspended after having been stored in dry form for a long time. This lag time delays the beneficial, infection-reducing effect provided by the probiotic microorganisms and reduces the medical value of the product.

The present inventor has discovered a new type of probiotic composition in which the probiotic microorganisms are present in an aqueous medium during storage and which has a shelf life of more than 3 month at 23 degrees C.

Thus, an aspect of the invention relates to a packaged probiotic composition, the probiotic composition comprising
a water-containing composition containing a viable first probiotic microorganism and
a support agent.

In some preferred embodiments of the invention, the support agent is in direct contact with the water-containing composition in the probiotic composition during storage. The support agent may for example have absorbed the water-containing composition or the probiotic composition may comprise a hydrogel which contains the support agent and the water-containing composition.

The inventor has discovered that for such embodiments, surprisingly, the stability of the support agent in the water-containing composition is important to obtain a long shelf-life of the probiotic composition. Furthermore, it may be desirable that the support agent does not degrade when applied to a wound. In such embodiments it is therefore preferred that the support agent is non-degradable by the water-containing composition.

Yet an aspect of the present invention pertains to a method of producing a packaged probiotic composition, the method comprising the steps of:

a) providing a water-containing composition containing a viable first probiotic microorganism,
b) providing a support agent,
c) optionally, contacting the support agent with the water-containing composition, and
d) packaging the combination of the support agent and the water-containing composition in a suitable primary container.

As stated above the support agent may in some embodiments of the invention be non-degradable by the water-containing composition.

Another aspect of the present invention pertains to a method of treating a human or animal subject having a colonized wound or tissue, or being at risk of having a colonized wound or tissue, the method comprising the steps of:

1) providing a packaged probiotic composition as described herein,
2) opening the primary container in which the probiotic composition has been packaged, and
3) applying the probiotic composition to the colonized wound or tissue.

Useful examples of animal subjects are domesticated animals such as e.g. cows, pigs, horses, sheep, goats, camels, cats, and dogs.

A further aspect of the invention pertains to a probiotic composition described herein for use in treatment or prevention of colonized wound or tissue.

### BRIEF DESCRIPTION OF THE FIGURES

FIG. 1 is a schematic illustration of a cross section of a pad wound dressing-type probiotic composition according to the invention,

FIG. 2a is a schematic illustration of a pad dressing-type probiotic composition (1) which contains a reservoir containing the water-containing composition (6) and a separate support agent (8),

FIG. 2b is a schematic illustration of the pad dressing-type probiotic composition (1) of FIG. 2a, wherein the water-containing composition of the reservoir (6) has been transferred the support agent (2),

FIG. 3a is a schematic illustration of a cross section of the components used to prepare an adhesive dressing-type probiotic composition,

FIG. 3b is a schematic illustration of an adhesive dressing-type probiotic composition (1),

FIG. 4a is a schematic illustration of an adhesive dressing-type probiotic composition (1) which contains a reservoir containing the water-containing composition (6) and a separate support agent (8), and

FIG. 4b is a schematic illustration of the adhesive dressing-type probiotic composition (1) of FIG. 4a, wherein the water-containing composition of the reservoir (6) has been transferred the support agent (9).

FIG. 5 is a schematic illustration of a packaged kit containing a sealed first secondary container (10) comprising the water-containing composition (11) and a second secondary container (13) comprising the support agent (8).

### DETAILED DESCRIPTION OF THE INVENTION

As mentioned, an aspect of the invention pertains to a packaged probiotic composition, the probiotic composition comprising
a water-containing composition containing a viable first probiotic microorganism, and
a support agent.

The probiotic composition may be a semi-homogeneous composition such as a hydrogel consisting essentially to the water-containing composition and the support agent, or it may be a structured composition containing a number of structural components in addition to the support agent and the water-containing composition. An example of such a structured probiotic composition is an adhesive wound dressing, which, in addition to the support agent and the water-containing composition, may contain a top layer to which the support agent and an adhesive layer are attached.

In the context of the present invention, the terms “consists essentially of” or “consisting essentially of” means that the process or product in question consists of the specified materials or steps and those that do not materially affect the basic and novel characteristic(s) of the invention.

In the context of the present invention, the term “packaged probiotic composition” pertains to a probiotic composition enclosed within a suitable container, e.g. a primary container, that serves to protect the probiotic composition from contamination from the environment. Preferably the primary container forms a barrier protecting the probiotic composition from the external non-sterile environment. The primary container is preferably first broken when the probiotic composition is used, for example as a wound dressing.

The water-containing composition comprises a first probiotic microorganism that is characterised as a viable microorganism, which when administered to a host can confer a health benefit by virtue of its ability to displace, inhibit and/or destroy a pathogenic microorganism. The probiotic microorganism has the capacity to inhibit and/or prevent colonization by a pathogenic microorganism in a wound or a tissue when applied to that wound or tissue.

In the context of the present invention, the phrase “Y and/or X” means “Y” or “X” or “Y and X”. Along the same line of logic, the phrase “n₁, n₂, . . . , nᵢ₋₁, and/or nᵢ” means “n₁” or “n₂” or . . . or “nᵢ₋₁” or “nᵢ” or any combination of the components: n₁, n₂, . . . nᵢ₋₁, and nᵢ.

The probiotic microorganism in the water-containing composition is viable and is thus at least capable of being metabolically active and preferably also capable of growing. The probiotic microorganism preferably has the capacity of metabolic activity and growth when used as a component of the probiotic composition and when brought in contact with one or more metabolisable substrates or nutrients present in exudate from the wound or tissue.

The water-containing composition is preferably a water-containing liquid and typically contains a significant amount of water. In preferred embodiments of the invention, the water-containing composition contains water in an amount of at least 50% (w/w) relative to the total weight of the water-containing composition.

For example, the water-containing composition may contain water in an amount of at least 60% (w/w), such as at least 70% (w/w), preferably at least 80% (w/w), and even more preferred at least 90% (w/w).

It may be preferred that the water-containing composition contains water in an amount of at least 95% (w/w), such as at least 97% (w/w).

The water-containing composition may for example contain an amount of water in the range of 50%-99.9% (w/w), such as in the range of 60%-99.7% (w/w), preferably in the range of 70%-99.5% (w/w), such as in the range of 80%-99.3% (w/w), and even more preferably in the range of 90%-99.1% (w/w) such as in the range of 95%-99% (w/w).

It should be noted that the water of the water-containing composition also encompasses water contained by the probiotic microorganism(s).

In preferred embodiments of the invention, the first probiotic microorganism is a bacterium or a fungus. It is particularly preferred that the first probiotic microorganism is a bacterium.

It is preferred that the bacterium is an anaerobic bacterium, for example a lactic acid producing bacterium such as e.g. a Lactobacillus species or a Bifodobacterium species.

In some preferred embodiments of the invention, the first probiotic microorganism is capable of producing one or more bacteriocin(s) (e.g subspecies of Lactobacillus lactic and Lactobacillus brevis). A bacteriocin-producing microorganism is advantageous in the context of the present invention, since bacteriocins are known as narrow spectrum antibiotics that act as proteinaceous toxins and inhibit the growth of other bacterial strains.

In some embodiments of the present invention, the probiotic microorganism is a lactic acid bacterium. The lactic acid bacterium may for example be a bacterium belonging to genus selected from the group consisting of a Carnobacterium, an Enterococcus, a Lactobacillus, a Lactococcus, a Leuconostoc, an Oenococcus, a Pediococcus, a Streptococcus, a Tetragenococcus, a Vaggococcus, a Weissella a and Bifodobacterium.

In some embodiments of the present invention, the first probiotic microorganism is a Bacillus strain (e.g. B. coagulans).

In other embodiments of the present invention, the first probiotic microorganism is a yeast, e.g. such as Saccharomyces boulardii, Saccharomyces cerevisiae or Aspergillus oryzae.

Other useful lactic acid bacteria and non-lactic acid bacteria may be found in WO 2008/074,331 or WO 00/61,201.

The water-containing composition may contain one or more additional probiotic microorganisms selected from the microorganisms mentioned above. The water-containing composition may for example contain a second probiotic microorganism, which is different to the first probiotic microorganism, and even a third probiotic microorganism which is different from the first and second probiotic microorganism.

Additionally, the water-containing composition may also contain one or more non-probiotic microorganisms (e.g. non-probiotic lactic acid bacteria).

However, it is preferred that neither the water-containing composition nor the probiotic composition as such contains pathogenic microorganisms.

In some preferred embodiments of the invention, the water-containing composition contains a total amount of probiotic microorganisms of at least about 10³ cfu (colony forming units) per mL. The probiotic composition may for example contain a total amount of probiotic microorganisms of at least about 10⁴ cfu, preferably at least 10⁵ cfu, such as at least 10⁶ cfu, or at least 10⁷ cfu, and even more preferably at least 10⁸ cfu, of probiotic microorganism per mL, such as at least 10⁹ cfu per mL.

Alternatively, the water-containing composition may contain a total amount of probiotic microorganisms between 10³ cfu and 10¹⁹ cfu per mL, preferably between 10⁵ cfu and 10⁹ cfu, and even more preferably between 10⁶ cfu and 10⁸ cfu, of probiotic microorganisms per mL. As will be understood by the person skilled in the art, the total amount of probiotic microorganisms encompasses all individual cells of the first probiotic microorganism as well as the individual cells of any additional probiotic microorganisms.

The present inventor has discovered that storage of packaged probiotic microorganisms sometimes is associated with undesirable gas production in the packaged probiotic composition. Even a moderate degree of gas production may lead to the formation of gas bubbles in the support agent or elsewhere in the probiotic composition, which may reduce the overall efficiency of the probiotic composition. A large degree of gas production is capable of expanding, and even exploding, the packaging in which the probiotic composition has been packaged, in which case the probiotic composition is rendered useless.

The present inventor has furthermore discovered that the problem associated with undesirable gas development can be solved by limiting to amount of low-molecular carbon-containing nutrients in the water-containing composition.

Thus, in some preferred embodiments of the invention, the total amount of carbon-containing nutrients having a molecular weight of at most 5000 g/mol in the water-containing composition is at most 0.5% (w/w) relative to the weight of the water-containing composition. For example, the total amount of carbon-containing nutrients having a molecular weight of at most 5000 g/mol in the water-containing composition may be at most 0.2% (w/w) relative to the weight of the water-containing composition, preferably at most 0.1% (w/w), and even more preferably at most 0.05% (w/w) relative to the weight of the water-containing composition, such as at most 0.001% (w/w).

In the context of the present invention, the term “carbon-containing nutrients having a molecular weight of at most 5000 g/mol” pertains to organic compounds that act as nutrients and carbon source to the first probiotic microorganism. Such compounds are e.g. alcohols, carbohydrates, peptides, fatty acids and combinations thereof. It should be noted that carbon-containing compounds, which have no nutritional value to the probiotic microorganism(s) are not perceived as carbon-containing nutrients according to the present invention.

Furthermore, the carbon-containing nutrients are preferably ones that are degraded and/or metabolised by the probiotic microorganism in the water-containing composition. The therapeutic efficacy of the probiotic microorganism in the probiotic composition is thought to depend on the metabolic activity of the microorganism, whereby the pH at the wound site is lowered to levels that inhibit proliferation of pathogenic organisms and inhibit proteolytic enzymes produced by such pathogens.

The viability of the first probiotic microorganism (or additional probiotic microorganisms) is pH dependent, and hence the shelf life of the packaged probiotic composition is extended if the water-containing composition has a pH of at most pH 6.0. For example, the water-containing composition may have a pH of at most pH 5, preferably at most pH 4.5, and even more preferably at most pH 4.

In one embodiment the water-containing composition of the packaged probiotic composition has a pH in the range of pH 2-6. For example, the water-containing composition may have a pH in the range of pH 2.5 to 5, preferably in the range of pH 3.0 to 4.5, or even more preferably in the range of pH 3.0 to 4.0.

Alternatively, the water-containing composition may have a pH in the range of pH 4 to 6, preferably in the range of pH 4.3 to 5.7, or even more preferably in the range of pH 4.7 to 5.3. It has been found that a pH in within these ranges reduces the patient discomfort and burning sensation which may arise when acidic substances are applied to a wound.

pH values mentioned in the context of the present invention pertain to the pH value of the relevant liquid at a temperature of 25 degrees C.

Additionally, the stability and/or viability of the probiotic microorganism is improved by keeping the probiotic microorganism in the same medium in which it has been fermented, and it is at least preferred that one or more of the fermentation product(s) is present in the water-containing composition.

Lactic acid is a fermentation product of lactic acid bacteria suitable for use in the packaged probiotic composition of the invention and may accordingly be present in the water-containing composition containing a first probiotic microorganism.

In some embodiments of the invention, the water-containing composition consists essentially of the fermentation broth obtained by fermenting the probiotic microorganism with carbon-containing nutrients until substantially all carbon-containing nutrients have been consumed. It is, however, possible to refine the fermentation broth. It may for example be preferred to remove components which are not physiologically acceptable, which interfere with the wound healing process or which inhibit the metabolism and/or growth of the probiotic microorganism once it has been applied to the wound or tissue to be treated.

Furthermore, lactic acid is also a suitable acid for regulating the pH of the water-containing composition.

In some embodiments the water-containing composition of the packaged probiotic composition comprises at least about 0.01% (w/w) lactic acid relative to the weight of the water-containing composition. For example, the water-containing composition may contain at least 0.05% (w/w) lactic acid, such as at least 0.1% (w/w), preferably at least 0.2% (w/w), such as at least 0.4% (w/w), and even more preferred at least 0.6% (w/w), such as at least 1% (w/w).

The water-containing composition may for example contain an amount of lactic acid in the range of 0.01%-2% (w/w), preferably in the range of 0.1%-1.5% (w/w), and even more preferably in the range of 0.2%-1% (w/w).

In the context of the present invention, the term “lactic acid” pertains to both the protonated and deprotonated state of lactic acid and therefore also encompassed lactate.

The water-containing composition, and the probiotic microorganisms contained therein, provides the rapid probiotic effect once the probiotic composition has been applied to the wound or tissue to be treated and thereby outcompete the pathogenic microorganisms of the wound or tissue.

The water-containing composition may for example be produced via a fermentation process, where probiotic microorganisms are mixed with water and nutrients and fermented at a suitable temperature, e.g. in the range of 20-45 degrees C., such as approx. 35 degrees C. Preferably, the fermentation continues until essentially all the carbon-containing nutrients have been consumed by the probiotic microorganisms.

The water-containing composition may furthermore contain other useful components such as salts, pH buffers and/or growth co-factors, which benefit the stability or the medical effect of the probiotic microorganism(s).

Examples of useful buffer agent are phosphate, lactic acid, acetic acid, and tartaric acid buffer. The phosphate buffer form part of a phosphate buffer saline solution.

The probiotic composition contains a support agent which preferably has an open, and e.g. porous, structure that serves as a structural support for the water-containing composition comprising the first probiotic microorganism. Alternatively, the support agent may be a gel network or may have gel forming properties and form a hydrogel in combination with the water-containing composition.

The thickness of the support agent is sometimes a relevant feature when the support agent is in the form of fibres, a foam, or gel network which forms a hydrogel sheet. In some embodiments of the invention the support agent has a thickness, i.e. the distance between the side of the support agent which is to face the wound or tissue and its opposite side, is at most 5 cm, such as e.g. at most 4 cm, or such as e.g. at most 3 cm. For example, the support agent may have a thickness which is at most 2 cm.

In some embodiments of the invention the support agent has a thickness in the range of 0.1-5 cm, preferably in the range of 0.2-4 cm, and even more preferably in the range of 0.5-3 cm. For example, the support agent may have a thickness in the range of 1-2 cm.

The support agent may be even thinner, and in some embodiments of the invention the support agent has a thickness in the range of 0.05-2 cm, preferably in the range of 0.1-1.5 cm, and even more preferably in the range of 0.2-1 cm. For example, the support agent may have a thickness in the range of 0.3-0.8 cm. The support agent may e.g. have a thickness in the range of 0.1-0.5 cm.

Furthermore, the support agent preferably has water-absorbing and/or water-retaining properties. In some preferred embodiment of the invention the support agent is water-insoluble. In the context of the present invention a support agent is deemed water-insoluble if it has a solubility of at most 0.5 g/100 g water at 23 degrees C.

It may for example be preferred that the support agent comprises, or even consists of, one or more solid material(s). The support agent may for example comprise, or even consist of, one or more polymer materials.

The support agent preferably comprises, or even consists of, one or more biocompatible material(s), and is therefore preferably both suitable for supporting the viable probiotic microorganism in the composition and suitable for contacting wounds or tissue.

In some embodiments of the invention, the support agent does not bind irreversibly to the microorganism and does not prevent growth and mobility of the microorganism in the composition. In preferred embodiments of the invention, the pore size of the support agent is at least 5 times higher than the average diameter of the probiotic microorganism(s), preferably at least 10 times higher, and even more preferably at least 20 times higher than the average diameter of the probiotic microorganism(s).

In other embodiments of the invention, the support agent binds at least some probiotic microorganisms irreversibly.

The support agent may for example comprise, or even consist of, one or more polymer material(s) selected from the group consisting of a polyester, a polypropylene, a polyethylene, a polyacrylate, a polyvinylpyrrolidone, a polyvinylalcohol, a polyurethane, a polyvinylacetate, a polysaccharide, and a combination thereof. The support agent may for example comprise, or even consist of, one or more polymer material(s) based on one or more monomers selected from the group consisting of a sodium acrylate, acrylic acid, methyl acrylate, methylmetacrylate, and a combination thereof.

Polymer materials used for the support agent may e.g. comprise, or even consist of, cross-linked polymers.

Materials, which are not inherently hydrophilic, e.g. polyethylene or polypropylenes, may have a modified surface which renders them hydrophilic. Alternatively, materials, which are not inherently hydrophilic, may be mixed with materials that are hydrophilic.

It is generally preferred that the support agent retains its shape, structure and/or colour during storage prior to use, ensuring an adequate shelf-life of the packaged probiotic composition.

The inventor has discovered that for such embodiments, surprisingly, the stability of the support agent in the water-containing composition is important to obtain a long shelf-life of the probiotic composition. Furthermore, it may be desirable that the support agent does not degrade when applied to a wound. In such embodiments it is therefore preferred that the support agent is non-degradable by the water-containing composition.

For embodiments of the present invention where the water-containing composition contacts the support agent during storage, it is preferred that the support agent is non-degradable by the water-containing composition. For example, support agents which do not contain hydrolysable backbone bonds tend to be non-degradable.

Such support agents are preferably formed by one or more polymer material(s) which do not contain a polymer backbone comprising a bond-type that is prone to hydrolysis under the pH present in the water-containing composition. The water-containing composition typically has a mild acidic pH (pH 2-6).

Additionally, enzyme from the probiotic microorganism, which may be present in the water-containing composition, can contribute to the degradation of the support agent. It is therefore preferred that such support agents are formed by one or more polymer material(s) which do not contain a polymer backbone comprising a bond-type that is prone to hydrolysis in the presence of the specific enzymes of the water-containing composition.

In the context of the present invention, the term “non-degradable support agent” means that the support agent only displays very limited degradation or preferably no degradation at all during long term exposure to the water-containing composition.

The degradability of the support agent depends on the actual pH of the water-containing composition and the enzymes contained therein. It may therefore be necessary to perform an assay to test whether a specific support agent is non-degradable by the water-containing composition.

However, support agents which typically are non-degradable by water-containing compositions described herein may e.g. comprise, or even consist of, one or more polymer material(s) selected from the group consisting of an acid stable polyester, a polyurethane, a polypropylene, a polyethylene, a polyacrylate, a polyvinylpyrrolidone, a polyvinylalcohol, a polyvinylacetate, and a combination thereof.

If the support agent is a gel network and the probiotic composition comprises an amorphous hydrogel comprising both the support agent and the water-containing composition, it is preferred that the probiotic composition looses at most 25% of its viscosity when heated to a temperature of 35 degrees C. for 3 days. The loss of viscosity is measured as:

$$\frac{{Π}_{before storage} - {Π}_{after storage}}{{Π}_{before storage}} * 100 \%$$

where n_{before storage} is the dynamic viscosity of the amorphous hydrogel before the above-mentioned 3 days storage and n_{after storage} is the dynamic viscosity of the amorphous hydrogel after the storage. The dynamic viscosity is measured on a Brookfield viscometer at 23 degree Celsius.

If the support agent in the form of fibres, a foam or a gel network forming a hydrogel sheet, a support agent is deemed non-degradable if it has a weight loss of at most 10% w/w (dry weight) when submerged in the water-containing composition and heated to a temperature of 35 degrees C. for 10 days.

The weight loss is measured as:

$$\frac{{w}_{before storage} - {w}_{after storage}}{{w}_{before storage}} * 100 \%$$

where w_{before storage} is the dry weight of the support agent before the above-mentioned 10 days storage and w_{after storage} is the dry weight of the support agent after the storage. The dynamic viscosity is measured on a Brookfield viscometer at 23 degree Celsius.

The weight loss determination is based on a sample of the support agent which has a dry weight of approx. 2 g and which has a maximum thickness of 2.0 cm. The dry support agent sample is weighed before storage to obtain w_{before storage}, and is then submerged in a volume of water-containing composition corresponding to 20 times the outer volume of the support agent sample.

After storage at a temperature of 35 degrees C. for 10 days, the support agent sample is separated from the water-containing composition, removing as much liquid as possible without damaging the support agent sample. Subsequently, the support agent sample is washed 3 times—each time with a volume of demineralised water corresponding to 20 times the outer volume of the support agent. The demineralised water should have a temperature of 23 degrees C. The support agent is contacted thoroughly with demineralised water and is allowed to stand for 20 minutes before as much water as possible is separated from the support agent. Once the demineralised water has been removed from the support agent, the next volume of clean demineralised water is brought in contact with the support agent sample as described above. When the support agent sample has been washed 3 times and most of the demineralised water has been removed, the moist support agent sample is placed in a heating cabinet providing a temperature of 70 degrees C. and a relative humidity of 25% and is then dried for 24 hours. Finally, the weight of the dry support agent support is measured to obtain w_{after storage}.

The support agent may for example comprise, or even consist of, one or more material(s) in a form selected from the group consisting of fibres, a foam, a gel network, a gel-forming agent, and a combination thereof.

In some embodiments of the invention, the support agent is present in dry form in the packaged probiotic composition, meaning that the support agent is not in contact with any substantial amounts of water.

For example, the support agent is deemed dry if the combination of the support agent and any water absorbed by the support agent contains at most 10% (w/w) water relative to the total weight of the support agent and absorbed water.

Support agents in dry form may for example be prepared by freeze-drying or vacuum-drying a support agent, or a mixture of support agents, in wet form.

In some embodiments of the invention, the support agent is present in wet form in the packaged probiotic composition, meaning that the support agent is in contact with liquid water. In this case, the combination of the support agent and any water absorbed by the support agent contains more than 10% (w/w) water relative to the total weight of the support agent and absorbed water, and preferably at least 20% (w/w) water, and even more preferably at least 30% (w/w) water.

In some preferred embodiments of the invention, the support agent comprises, even consists of, fibres. Such fibres may for example be in the form of fabrics of woven fibres, non-woven fibres, or a combination thereof. The fibres may e.g. comprise, or even consist of, polymers such as polyester, polyurethane, polypropylene, polyethylene, polyacrylate, absorbent polyacrylate and a combination thereof.

The fibres may e.g. be attached to a polymer film, e.g. a polyester or polyurethane film.

A preferred type of fibres is super absorbent fibres (SAF) which may comprise, or even consist of, acrylic fibres. SAF fibres often have poor wet strength, why, when using SAF, it may be advantageous to blend the SAF with fibres that has a higher wet strength, such as e.g. polyester fibres.

In some embodiments of the invention, the fibres of the support agent comprise 25-95% (w/w) super absorbent fibres, preferably 35-85% (w/w), and even more preferably 50-80% (w/w) (w/w) super absorbent fibres relative to the total weight of fibres used in the support agent.

In some preferred embodiments of the invention the support agent comprises a mixture of superabsorbent fibres and low absorbent fibres.

In some preferred embodiments of the invention the support agent comprises fibres having a low or non-water absorbent core and a water absorbent shell. Such fibres may e.g. be Lanseal fibres from Toyobo, JP.

In the context of the present invention, the term “super absorbent fibre” means fibres that absorb at least 25 gram purified water per gram fibre.

In the context of the present invention, the term “low absorbent fibre” means fibres that absorb at most 10 gram purified water per gram fibre.

In some preferred embodiments of the invention, the support agent comprises, or even consists of, a foam.

A foam may e.g. comprise, or even consist of, polymers such as e.g. polyvinylalcohol, polyacrylate, polyurethane, polyvinylacetate or a combination thereof.

In some preferred embodiments of the invention, the support agent comprises, or even consists of, a gel-forming agent.

In the context of the present invention, the term “gel-forming agent” pertains to agents which will form a hydrogel or a hydrogel-like viscous composition upon contact with the water-containing composition.

Useful hydrogel-forming agents typically include polyvinylpyrrolidone, polyvinylalcohol, polyacrylate, polyurethane, polyvinylacetate and the like.

In some preferred embodiments of the invention, the support agent comprises, or even consists of, a gel network.

In the context of the present invention, the term “gel network” pertains to the network of cross-linked gel-forming agents, which form the skeleton of a hydrogel.

In some preferred embodiments of the invention, the probiotic composition comprises, or even consists of, a hydrogel comprising the support agent and water. In this case the support agent comprises, or even consists of, the gel network.

A hydrogel may take the form of a hydrogel sheet i.e. a hydrogel containing a continuous matrix of cross linked polymers, thereby forming a sheet-like hydrogel-structure. In this case the gel network typically is continuous network of cross-linked gel-forming agents.

Alternatively, the hydrogel may take the form of an amorphous hydrogel. In this case the gel network typically contains numerous network fragments of cross-linked gel-forming agents. When hydrated, the network fragments can move relative to each other and there provide the amorphous hydrogel with liquid-like properties.

In yet an alternative, the hydrogel may be a highly viscous liquid having properties similar to an amorphous hydrogel.

Where the probiotic composition as such is an amorphous hydrogel comprising the first probiotic microorganism, or where the support agent forms an amorphous hydrogel with the water-containing composition, the amorphous hydrogel may have a dynamic viscosity of at least 50000 cP. For example, the amorphous hydrogel may have a dynamic viscosity of at least 1*10⁵ cP, preferably at least 3*10⁵ cP, such as at least 6*10⁵ cP, and even more preferably at least 9*10⁵ cP, such as at least 1*10⁶ cP. The dynamic viscosity is measured on a Brookfield viscometer at 23 degree Celsius.

Alternatively, the amorphous hydrogel may have a dynamic viscosity in the range of 5*10⁴-2*10⁶ cP, preferably in the range of 1*10⁵-1.5*10⁶ cP, and even more preferably in the range of 5*10⁵-1*10⁶ cP.

Where the packaged probiotic composition comprises a hydrogel comprising the support agent and the water-containing composition, the hydrogel preferably contains at least 50% (w/w) water. Alternatively, the hydrogel may contain at least 60% (w/w), such as at least 70% (w/w), preferably at least 80% (w/w), such as at least 90% (w/w), or even more preferably at least 95% (w/w), such as at least 97% (w/w) water. Alternatively, the hydrogel may contain between about 50%-60% (w/w) water, or 60%-70% (w/w) water, or 70%-80% (w/w) water, or 80%-90% (w/w) water, 90%-97% (w/w) water or 95%-99% (w/w) water.

In some preferred embodiments of the invention, the probiotic composition is a wound dressing.

In some embodiments of the invention, where the probiotic composition is a wound dressing, the support agent comprises, or even consists of, one or more material(s) in a form selected from the group consisting of fibres, a foam, a gel network, or gel-forming agent, and a combination thereof.

The probiotic composition may for example be a so-called pad dressing, which comprises, or even consists of, the support agent, the water-containing compositions, and optionally also one or more additional layers such as moisture barrier layer. Pad dressings typically need a bandage or other means to keep them in place on the treatment site.

Preferred support agents for a pad dressing may contain one or more material(s) in the form of fibres, a foam, or a gel network. The support agent for the pad may furthermore contain gel forming agents—particularly when the support agent is used in dry form.

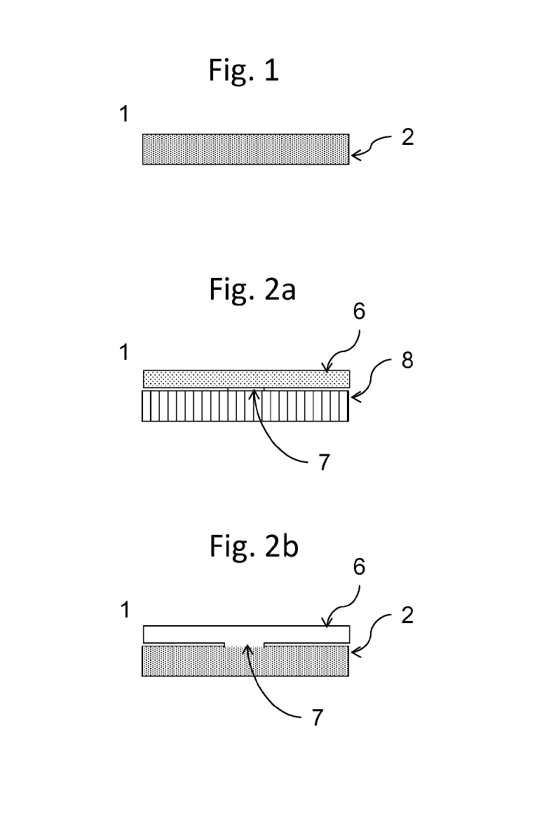

An exemplary embodiment of a probiotic composition (1) which is a pad wound dressing is illustrated in FIG. 1, which contains a schematic illustration of a cross section of a support agent containing the water-containing composition (2).

In some preferred embodiments of the invention, the water-containing composition contacts the support agent. This is for example the case when the probiotic composition is a hydrogel or when the probiotic composition comprises, or even consists of, a wound dressing soaked with the water-containing composition.

However, in other preferred embodiments of the invention, the water-containing composition does not contact the support agent while the probiotic composition is in a first state, but where the probiotic composition can be transformed to a second state where the water-containing composition contacts the support agent.

The first state is typically the state where the probiotic composition during storage is in the unopened primary container. The transformation to the second state preferably occurs during or after the opening of the primary container, or immediately before opening the primary container.

In some embodiments of the invention, the probiotic composition comprises a sealed reservoir containing the water-containing composition and a conduit which will allow the water-containing composition to contact the support agent when the sealed reservoir is opened. The sealed reservoir is typically opened during the above-mentioned transformation.

An exemplary embodiment of this is schematically illustrated in FIGS. 2a and 2b. In addition to the components described in FIG. 1, this probiotic composition furthermore contains a reservoir (6) in which the water-containing composition is sealed during storage. The reservoir (6) is preferably connected to the support agent (8) via a conduit (7). In FIG. 2a, the probiotic composition is in its first state, i.e. the water-containing composition is kept in the reservoir (6), separate from the support agent (8). The probiotic composition of this embodiment can be transformed into a second state, schematically illustrated in FIG. 2b, where the water-containing composition of the reservoir (6) has been transferred to the support agent (2) which now contains both water and first probiotic microorganisms from the water-containing composition.

The transformation typically involves opening the reservoir (6) and allowing the water-containing composition to flow via the conduit (7) into the support agent (8).

The reservoir may e.g. be opened by pressing part of the reservoir against a protrusion which perforates the reservoir wall and allows the water-containing composition to flow into the support agent. Alternatively, the reservoir may be designed to break in a controllable fashion when it is bent, and the breakage allows the water-containing composition to flow to and be absorbed by the support agent.

The reservoir may furthermore comprise an agitation mechanism as defined herein.

The packaged probiotic composition, when used as a wound dressing, may furthermore comprise an adhesive layer that serves to form a contact layer between the skin surrounding the wound, or the wound as such, and the applied dressing. The adhesive layer is preferably permeable or semi-permeable allowing exudate to evaporate out of the dressing. Preferred adhesive layers comprise absorbent adhesives (such as Comfeel transparent dressing from Coloplast A/S, Denmark), silicone adhesives, polyacrylate adhesives or polyurethane adhesives.

The probiotic composition may furthermore comprise an adhesive layer. Such an adhesive layer should preferably be capable of adhering the probiotic composition to the site where it is to be active.

A top layer may form the outer surface of the probiotic composition most remote from the wound contact surface that serves to prevent leakage of wound exudate from the dressing and to prevent entry of contaminants into the wound dressing. Preferably the top layer is able to breathe, being permeable to both oxygen and to moist vapour. A vapour transmission rate of at least 1000 grams per day per square meter per 24 hours is preferred. Suitable materials for the top layer are polyurethane films.

In one embodiment, the water-containing composition is not in contact with the support agent prior to use (i.e in its unopened state prior to use), and where the step of opening the packaging causes a transformation of the probiotic composition when the water-containing composition contacts the support agent.

The support agent may for example be attached to the top layer, either directly or indirectly, through one or more other materials.

The probiotic composition may furthermore comprise a bottom layer. A bottom layer is preferably highly water permeable and preferably porous. In some embodiments of the invention the bottom layer has pores or openings which are sufficiently large to allow the water-containing composition including the first probiotic microorganism to flow though the bottom layer into the wound or tissue to be treated. However, it may be preferred that the pores or openings are sufficiently small to retain the support agent. This embodiment is particularly preferred when the support agent must not contact the wound or tissue to be treated.

In some preferred embodiments of the invention, the probiotic composition comprises a top layer, a bottom layer and an adhesive, and wherein said support agent and said water-containing composition are located in a cavity which is at least partly defined by the top layer and/or the bottom layer, and wherein the adhesive is arranged such that the probiotic composition can adhere to a skin surface in such a way that the bottom layer contacts the wound surface.

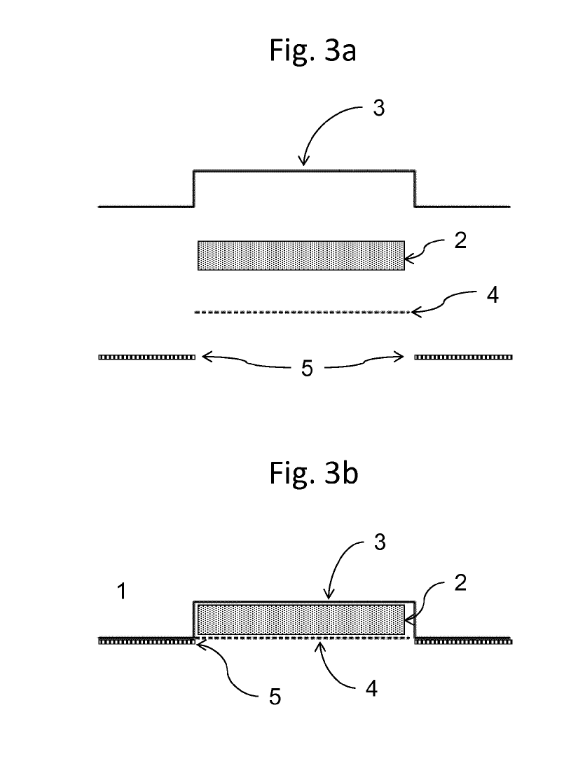

An exemplary embodiment of this is shown in FIGS. 3a and 3b. FIG. 3a shows the components used to produce the probiotic composition: A top layer (3), a support agent which contains, and contacts, the water-containing composition (2), a bottom layer (4) and an adhesive layer (5). The probiotic composition may be produced by placing the support agent (2), which contains the water-containing composition, in a cavity formed by the top layer (3) and closing the cavity by attaching the bottom layer (4) to at least a part of the top layer (3), which part surrounds the cavity. The attachment may be performed by use of traditional attachment techniques, such as welding or use of an adhesive.

Additionally, if the top layer does not have sufficient adhesive characteristics itself, an adhesive layer (5) may be present on at least some of the parts of surface the top layer which is to contact the skin surrounding the wound or tissue to be treated. The adhesive layer (5) may furthermore be attached to a non-sticking sheet (not shown in the figures) which prevents the adhesive layer (5) from sticking to the packaging of the packaged probiotic composition, and which has to be removed from the probiotic composition prior to use.

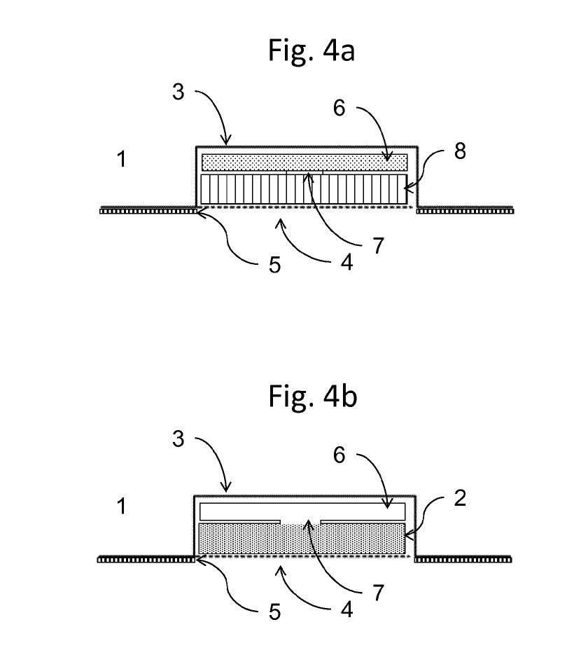

An exemplary embodiment of this is schematically illustrated in FIGS. 4a and 4b. In addition to the components described in FIGS. 3a and 3b, this probiotic composition furthermore contains a reservoir (6) in which the water-containing composition is sealed during storage. The reservoir (6) is preferably connected to the support agent (8) via a conduit (7). In FIG. 3a, the probiotic composition is in its first state, i.e. the water-containing composition is kept in the reservoir (6), separate from the support agent (8). The probiotic composition of this embodiment can be transformed into a second state, schematically illustrated in FIG. 4b, where the water-containing composition of the reservoir (6) has been transferred to the support agent (2) which now contains both water and first probiotic microorganisms from the water-containing composition.

The transformation typically involves opening the reservoir (6) and allowing the water-containing composition to flow via the conduit (7) into the support agent (8).

As stated above, the reservoir may e.g. be opened by pressing part of the reservoir against a protrusion which perforates the reservoir wall and allows the water-containing composition to flow into the support agent. Alternatively, the reservoir may be designed to break in a controllable fashion when it is bent, and the breakage allows the water-containing composition to flow to and be absorbed by the support agent.

The probiotic composition preferably contains sufficient water to be able of donate water to the wound or tissue to be treated.

In one embodiment, the probiotic composition contains at least 0.1 mL of the water-containing composition. For example, the probiotic composition may contain at least 0.5 mL of the water-containing composition, such as at least 1 mL, or even at least 3 mL of the water-containing composition.

Even higher amounts of amounts of water-containing composition may be used. Thus in some embodiments of the invention, the probiotic composition contains at least 50 mL of the water-containing composition. For example, the probiotic composition may contain at least 100 mL of the water-containing composition, such as at least 200 mL, or even at least 300 mL of the water-containing composition.

In some preferred embodiments of the invention, the probiotic composition contains in the range of 0.1-100 mL of the water-containing composition, preferably in the range of 0.2-50 mL, preferably in the range of 0.5-30 mL, and even more preferably in the range of 1-20 mL of the water-containing composition.

Even higher amounts of amounts of water-containing composition may be used. Thus in some embodiments of the invention, the probiotic composition contains an amount of the water-containing composition in the range of 50-500 mL. For example, the probiotic composition may contain an amount of the water-containing composition in the range of 100-450 mL, preferably in the range of 150-400 mL, or even in the range of 200-350 mL.

An advantage of the probiotic composition of the present invention is that it is particularly suited for dry wounds as it both donates water to the wound and provides a controlled microbial environment containing probiotic microorganisms.

In some preferred embodiments, the support agent of the packaged probiotic composition may have absorbed an amount of water, e.g. provided by the water-containing composition, corresponding to at least 50% of the water holding capacity of the support agent. For example, the support agent may contain an amount of water corresponding to at least 75% of its water holding capacity, preferably at least 90%, and even more preferred at least 95% its water holding capacity. In embodiments where the support agent is kept separate from the water-containing composition during storage, the above water contents describe situations where the support agent has been contacted by the water-containing composition prior to the application of the probiotic composition to the wound or tissue.

The water holding capacity is measured according to EN 13726-1 section 3.2 where a given weight of wound dressing is submerged into saline solution followed by a weight measurement.

In some preferred embodiments of the invention the weight ratio between the support agent and the water-containing composition is at most 1:1, preferably at most 1:2, and even more preferred at most 1:5. For example, the weight ratio between the support agent and the water containing composition may be at most 1:10, preferably at most 1:20, and even more preferred at most 1:40, such as approx. 1:50.

In some preferred embodiments of the invention the weight ratio between the support agent and the water-containing composition is in the range of 1:1-1:50, preferably in the range of 1:2-1:30, and even more preferably in the range of 1:4-1:20.

The probiotic composition has been packaged in a suitable primary container and is therefore a packaged probiotic composition. The primary container is important for maintaining a controlled microbial environment in the probiotic composition and for keeping the water in the water-containing composition.

The primary container in which the probiotic composition is packaged, typically includes a material which provides a protective sterile barrier layer having a water permeability of at most 10 g/m²/24 hours (measured according to EN 13726-2, section 3.2). For example, the material of the primary container may provide a barrier layer having a water permeability of at most 5 g/m²/24 hours. Alternatively, the material of the primary container may provide a barrier layer having a water permeability of at most 1 g/m²/24 hours.

Suitable materials are typically selected from plastics, aluminium foil, plastic laminates, optionally bonded with an adhesive (e.g. polyurethane). Suitable plastics include: PET, PE, LLDPE, CPP, PA, PETP, METPET and Tyvek. If the water-containing composition and the support agent already have been packed in secondary containers, the primary container may be a cardboard- or paper-based container.

Several container types may be useful for packaging the probiotic composition. Non-limiting examples of useful containers are welded films such as aluminum-films, boxes, bags, trays, cans, and wrappers.

An advantage of the present invention is that the packaged probiotic composition has a very long shelf-life. In some embodiments the packaged probiotic composition has a shelf-life of at least 6 month when kept at a temperature of 23 degrees C. and a relative humidity of 50%. For example, the packaged probiotic composition may have a shelf-life of at least 9 month, such as at least 12 months, and even more preferably at least 18 months when kept at a temperature of 23 degrees C. and a relative humidity of 50%.

The shelf life at a given temperature and humidity is determined as the time running from production of the packaged probiotic composition to the first occurrence of at least one of the following events during storage:
Gas bubbles can be observed by visual inspection of the probiotic composition
The probiotic composition has lost more than 99% of the viable probiotic microorganisms relative to the number of colony forming units (cfu) of probiotic microorganisms present in the newly produced probiotic composition,
Degradation of the support agent can be observed by visual inspection, and
Discolouration of the support agent can be observed by visual inspection.

In some preferred embodiments of the invention, the packaged probiotic composition is a packaged kit containing
a sealed first secondary container comprising the water-containing composition, and
the support agent.

The support agent may be present in the kit as such or it may be present in a a sealed second secondary container which contains the support agent.

The first and second secondary containers may both be contained by the primary container. The primary container may for example comprise a cavity wherein both the first and second secondary containers are present, or the primary container may comprise two cavities which each contain the first or the second secondary container.

Thus an aspect of the invention relates to a packaged kit containing
a sealed first secondary container comprising a water-containing composition, and
a support agent.

In some preferred embodiments of the invention the sealed first secondary container is not attached to the sealed second secondary container, or it is attached to the sealed second secondary container so that the two containers can be separated from each other without opening the second sealed contain. Such a separable type of attachment can e.g. be obtained by adhering the first secondary container to the second secondary container using a suitable adhesive.

In another embodiments of the invention part of the material that forms part of the first secondary container also forms part of the second secondary container.

The kit may furthermore comprise a fixation member to fix the support agent to the wound. Useful examples of fixation members are e.g. a non-adhesive bandage or an adhesive component which is capable of attaching the support agent to the skin or wound of the subject to be treated. The adhesive components may for example be an adhesive tape. The non-adhesive bandage may e.g. be a gauze bandage or similar bandages.

The fixation member may be located in a third secondary container comprised by kit or it may be comprised by the kit as such.

In some embodiments of the invention, the adhesive component is attached to the support agent and the combination of the support agent and the adhesive component is located in the sealed second secondary container.

In some embodiments of the invention, the fixation member is capable of absorbing excess liquid from the support agent.

The secondary containers are sealed in the sense that no dirt or microorganisms from outside can get in to the water-containing composition or the support agent.

The sealed first secondary container contains the water-containing composition and should therefore be sufficiently water tight to avoid leakage of substantial amounts of water from the water-containing composition.

Thus, in some embodiments of the invention the first secondary container loses at most 10% (w/w) of its water during 180 days of storage at 23 degrees C. and at a relative humidity of 50%. For example, the first secondary container may lose at most 5% (w/w) of its water during 180 days of storage at 23 degrees C. and at a relative humidity of 50%. Alternatively, the first secondary container may lose at most 3% (w/w) of its water during 180 days of storage at 23 degrees C. and at a relative humidity of 50%.

In some preferred embodiments of the invention the first secondary container loses at most 2% (w/w) of its water during 180 days of storage at 23 degrees C. and at a relative humidity of 50%, and preferably even less.

As described above, containers having a low loss of water during storage are preferably produced using one or more materials having a low water permeability. Such materials are well-known in the art.

The present inventors have discovered that the actual design of the first secondary container has a significant impact on the usability of the probiotic composition.

The first secondary container preferably comprises an opening mechanism that allows for controlled opening of the first secondary container.

Non-limiting examples of useful opening mechanisms are a cap to be pealed or screwed off a bottle, a seal to be removed, a part of the first secondary container to be removed, e.g. by breaking or tearing.

In some embodiments of the invention, the activation of the opening mechanism opens a passage through which the water-containing composition may leave the first secondary container. Thus, after the opening mechanism has been activated the water-containing composition is in fluid communication with the exterior of the first secondary container.

While the passage may differ in size and cross section, it is presently preferred that the cross section of the passage is so that gravity alone cannot force the water-containing composition out of the first secondary container.

In some embodiments of the invention, the effective inner diameter of the passage is at most 5 mm, preferably at most 4 mm, and even more preferably at most 2 mm. The effective inner diameter of the passage may for example be at most 1 mm, preferably at most 0.5 mm, and even more preferably at most 0.2 mm.

In the context of the present invention, the term “effective inner diameter” is the diameter for the largest circle that can be drawn inside the cross section of the passage.

It is particularly preferred that the person using the first secondary container can control when the water-containing composition is released from the opened first secondary container. Furthermore, it may be preferred that the person using the opened first secondary container controls how much of the water-containing composition that is released.

The first secondary container may contain one or more visual indicators showing how much of the water-containing composition that corresponds to one or more pre-determined amounts of the water-containing composition. The pre-determined amount may e.g. relate to the weight of the predetermined amount or the volume of the predetermined amount.

For example, the first secondary container may comprise a visual indicator marking half of the total amount of the water-containing composition. Alternatively, the first secondary container may comprise visual indicators marking a quarter, half, and three quarters of the total amount of the water-containing composition. The first secondary container may comprise visual indicators marking each dL of the total amount of the water-containing composition.

The first secondary container may comprise visual indicators marking how much of the water-containing composition that remains in the first secondary container or alternatively how much of the water-containing composition that has been used.

A first secondary container which contains one or more of the above-mentioned visual indicators preferably has one or more transparent or semi-transparent areas through which the user can assess the remaining level water-containing composition.

Non-limiting examples of first secondary containers which contain one or more of the above-mentioned visual indicators are a transparent or semi-transparent glass bottle or a transparent or semi-transparent syringe which is volumetric graduated.

In some preferred embodiments of the invention, the water-containing composition is transferred from the opened first secondary container by exerting a pressure on at least a part of the outer surface of the first secondary container. For example the first secondary container may be a soft bottle or pouch having a narrow passage, which passage is closed during storage. In the opened first secondary container, the passage is also open but the water-containing composition does not leave the container before a pressure is exerted on at least a part of the outer surface of the first secondary container.

Non-limiting examples of exerting a pressure on the first secondary container is by pressing or squeezing the first secondary container.

As will be apparent from the above, the first secondary container may be shaped in many different ways and different materials.

In some embodiments of the invention, the first secondary container is a bottle-like container made of a soft, flexible material, e.g. an organic polymer, that can be pressed by hand to empty the first secondary container once it has been opened. The bottle-like container preferably has narrow passage that does not allow for outflow of water-containing composition and inflow of air at the same time.

In other embodiments of the invention, the sealed first secondary container is a pouch filled with the water-containing composition. The pouch preferably contains a narrow passage as defined above which allows for controlled transfer of water-containing composition to the support agent.

In some embodiments of the invention, the first secondary container is a syringe, preferably made of one or more materials having low water permeability. During storage, the tip of the syringe is sealed, e.g. by a cap which is glued to or pressed over the tip. In this case, the opening mechanism is the cap which should be removed to open the passage in the tip of the syringe.

The water-containing composition is sealed inside the syringe between the syringe walls, the pistol and the cap. The water-containing composition is transferred from the syringe by removing the cap from the tip of the syringe and pressing the piston further into the syringe.

In other embodiments of the invention the first secondary container is a bootle or bottle-like container having as passage an opening from where the water-containing composition can be poured onto the support agent. The bottle is furthermore fitted with an opening mechanism such as cap which can be unscrewed to open the bottle or bottle-like container. Alternatively, the opening mechanism of the bottle or bottle-like container may be e.g. a seal which closes the passage of the bottle or bottle-like container, and which seal can be removed, broken or otherwise modified to open the passage. When the water-containing composition is transferred from the first secondary container to the support agent by pouring the water-containing composition out of the water-containing composition, the effective inner diameter of passage is preferably sufficiently large to allow for simultaneous outflow of water-containing composition and inflow of air.

In some embodiments of the invention the effective inner diameter of the passage is at least 0.6 mm, preferably at least 1 cm, and even more preferably at least 1.5 cm. The effective inner diameter of the passage may for example be at least 2 cm, preferably at least 2.5 mm, and even more preferably at least 3 mm.

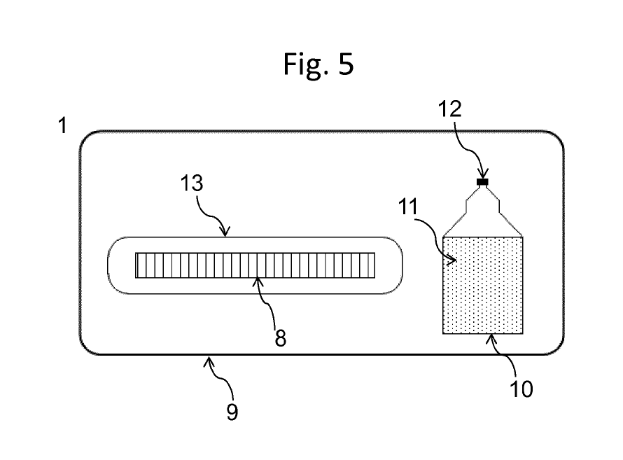

An exemplary embodiment of the invention is depicted schematically in FIG. 5, wherein the packaged probiotic composition (1) is a kit located in a primary container (9). The kit comprises a bottle-like first secondary container (10) containing the water-containing composition (11). The first secondary container (10) is sealed with a small cap (12), which can be removed to open the sealed first secondary container (10). The kit furthermore comprises second secondary container which comprises the support agent.

The first secondary container may be produced by conventional techniques and it is preferably sterilized before the water-containing composition is introduced. Likewise, the sealing of the first secondary container may be performed by conventional sealing techniques such as welding, gluing, attachment of a cap, or zipping the first secondary container using an integrated zipper.

Useful materials for the first secondary container preferably have a low water permeability and may e.g. be one or more of those mentioned herein context of materials of the primary container. If the opening mechanism of the first secondary container is a cap or a cap-like structure it may comprise an aluminum cap equipped with a liner of an expanded organic polymer, such as e.g. expanded polyethylene (EPE) or expanded polypropylene (EPP).

The sealed second secondary container contains the support agent, preferably the support agent in dry state. The sealed second secondary container forms a barrier between the support agent, which preferably is sterile, and the surroundings.

The second secondary container may be produced, filled and sealed using conventional techniques for such purposes.

The support agent for a kit may be any of the support agents described herein. The support agent for a kit preferably has an open, and e.g. porous, structure that serves as a structural support for the water-containing composition comprising the first probiotic microorganism.

Furthermore, the support agent preferably has water-absorbing and/or water-retaining properties. In some preferred embodiment of the invention the support agent is water-insoluble.

The support agent may e.g. comprise, or even consist of, materials such as e.g. fibres, a foam, or a gel network which forms a hydrogel sheet upon contact with water, or a combination thereof. Fibres may for example be in the form of fabrics of woven or non-woven fibres, or a combination thereof.

In some preferred embodiments of the invention, the support agent of the kit is a dry fabric, and preferably dry non-woven fabric.

The thickness of the support agent for kits is sometimes a relevant feature when the support agent is in the form of fibres, a foam, or gel network which forms a hydrogel sheet. In some embodiments of the invention the support agent has a thickness, i.e. the distance between the side of the support agent which is to face the wound or tissue and its opposite side, is at most 5 cm, such as e.g. at most 4 cm, or such as e.g. at most 3 cm. For example, the support agent may have a thickness which is at most 2 cm.

In some embodiments of the invention the support agent has a thickness in the range of 0.1-5 cm, preferably in the range of 0.2-4 cm, and even more preferably in the range of 0.5-3 cm. For example, the support agent may have a thickness in the range of 1-2 cm.

The support agent may be even thinner, and in some embodiments of the invention the support agent has a thickness in the range of 0.05-2 cm, preferably in the range of 0.1-1.5 cm, and even more preferably in the range of 0.2-1 cm. For example, the support agent may have a thickness in the range of 0.3-0.8 cm. The support agent may e.g. have a thickness in the range of 0.1-0.5 cm.

The support agent may be provided in various shapes and sizes. The support agents for kits are typically sheet-like support agents. The shape of such a sheet-like support agent may for example be rectangular, square, oval, or circular. Preferably, the user or a medical professional is able to adapt the size of the support agent to the wound that needs to be treated, e.g. by cutting the support agent with a pair of scissors.

In some preferred embodiments of the invention, the support agent covers a skin area of at least 1 cm². Preferably, the support agent covers a skin area of at least 10 cm². Even more preferably the support agent covers a skin area of at least 25 cm².

Even larger support agents may be required for large wounds. Thus, in some preferred embodiments of the invention, the support agent covers a skin area of at least 50 cm². Preferably, the support agent covers a skin area of at least 75 cm². Even more preferably, the support agent covers a skin area of at least 100 cm².

In some preferred embodiments of the invention, the support agent is capable of covering a skin area in the range of 1-500 cm². Preferably, the support agent is capable of covering a skin area in the range of 10-400 cm². Even more preferably the support agent is capable of covering a skin area in the range of 25-300 cm².

In some preferred embodiments of the invention, the support agent has a rectangular shape having the side lengths in the range 3-30 cm*5-30 cm. For example, the support may agent have a rectangular shape having the side lengths in the range 5-20 cm*5-20 cm. The support agent may e.g. have a rectangular shape with side lengths of about 10 cm*10 cm. Alternatively, the support agent may have a rectangular shape with side lengths of about 10 cm*20 cm. A larger support agent may e.g. have a rectangular shape with side lengths of about 20 cm*20 cm. A relatively small support agent may e.g. have a rectangular shape with side lengths of about 5 cm*5 cm.

The amount of water-containing composition in the sealed first secondary container may be selected to be in the same order of magnitude as the maximum water absorption of the support agent.

For example the amount of water-containing composition in the sealed first secondary container may be at least 25% of the water holding capacity of the support agent. The amount of water-containing composition in the sealed first secondary container may e.g. be at least 50% of the water holding capacity of the support agent. For example the amount of water-containing composition in the sealed first secondary container may be at least 70% of the water holding capacity of the support agent. Alternatively, the amount of water-containing composition in the sealed first secondary container may e.g. be at least 90% of the water holding capacity of the support agent. It may e.g. be preferred that the amount of water-containing composition in the sealed first secondary container is approx. 100% of the water holding capacity of the support agent.

The amount of water-containing composition in the sealed first secondary container may e.g. be in the range of 25-200% of the water holding capacity of the support agent. For example, the amount of water-containing composition in the sealed first secondary container may be in the range of 25-150% of the water holding capacity of the support agent. Alternatively, the amount of water-containing composition in the sealed first secondary container may be in the range of 50-150% of the water holding capacity of the support agent.

Even though the amount of water-containing composition of the sealed first secondary container may be much higher than the water holding capacity of the support agent, it is sometimes preferred that the amount of water-containing composition in the sealed first secondary container is at most 200% of the water holding capacity of the support agent. For example, the amount of water-containing composition in the sealed first secondary container may e.g. be at most 150% of the water holding capacity of the support agent. Alternatively, the amount of water-containing composition in the sealed first secondary container may e.g. be at most 125% of the water holding capacity of the support agent.

The amount of water-containing composition in the sealed first secondary container may e.g. be in the range of 100-200% of the water holding capacity of the support agent, and preferably in the range of 100-150% of the water holding capacity of the support agent.

It is normally preferred that substantially all of the water-containing composition of the first secondary container can be transferred to the support agent. If the first secondary container contains some water-containing composition, which cannot be transferred to the support agent the abovementioned amounts of water-containing composition should be increased accordingly.

In some preferred embodiments of the invention, the first secondary container contains an amount of water-containing composition, which is in the range of 80-120% (w/w) of the intended dosage of the water-containing composition for the support agent. For example, the first secondary container may contain an amount of water-containing composition, which is in the range of 90-110% (w/w) of the intended dosage of the water-containing composition for the support agent. The first secondary container may e.g. contain an amount of water-containing composition, which is in the range of 95-105% (w/w) of the intended dosage of the water-containing composition for the support agent.

In some preferred embodiments of the invention, the sealed first secondary container is intended to be used only once and is therefore a disposable container.

Some probiotic microorganisms tend to settle, at least to some extent, over time in the sealed first secondary container, which may lead to a reduction of the effective concentration of probiotic microorganisms that will be brought to the wound.

In some preferred embodiments of the invention, the sealed first secondary container contains a mechanism for agitating the water-containing composition before use, e.g. before opening the first secondary container, so as to obtain a more even distribution of probiotic microorganisms.

In some preferred embodiments of the invention, the mechanism for agitating the water-containing composition is one or more bubbles or pockets of gas, e.g. air or an inert gas, located inside the first secondary container and contacting the water-containing composition. When the sealed first secondary container is shaken by hand or turned upside down one or more times the one or more bubbles or pockets of gas move through the water-containing composition and thereby create agitation, which distributes the probiotic microorganisms more evenly in the water-containing composition.

In some preferred embodiments of the invention, the first secondary container contains at least 1% (vol/vol) gas in the form of one or more gas pocket(s) and/or gas bubble(s) relative to the total inner volume of the first secondary container. For example, the first secondary container may contain at least 5% (vol/vol) gas in the form of one or more gas pocket(s) and/or gas bubble(s) relative to the total inner volume of the first secondary container. The first secondary container may e.g. contain at least 10% (vol/vol) gas in the form of one or more gas pocket(s) and/or gas bubble(s) relative to the total inner volume of the first secondary container.

The first secondary container may contain an amount of gas in the form of one or more gas pocket(s) and/or gas bubble(s) in the range of 1-50% (vol/vol) relative to the total inner volume of the first secondary container. For example, the first secondary container may contain an amount of gas in the form of one or more gas pocket(s) and/or gas bubble(s) in the range of 5-40% (vol/vol) relative to the total inner volume of the first secondary container. The first secondary container may e.g. contain an amount of gas in the form of one or more gas pocket(s) and/or gas bubble(s) in the range of 10-30% (vol/vol) relative to the total inner volume of the first secondary container.

The first secondary container may e.g. contain at least 0.5 mL gas in the form of one or more gas pocket(s) and/or gas bubble(s). For example, the first secondary container may e.g. contain at least 1 mL gas in the form of one or more gas pocket(s) and/or gas bubble(s). Alternatively, the first secondary container may e.g. contain at least 5 mL gas in the form of one or more gas pocket(s) and/or gas bubble(s), such as e.g. at least 10 mL gas.

In other preferred embodiments of the invention, the mechanism for agitating the water-containing composition is one or more unattached solid bodies, e.g. one or more glass, ceramic, plastic or metal bodies, located inside the first secondary container and contacting the water-containing composition. When the sealed first secondary container is shaken by hand or turned upside down one or more times, the one or more unattached solid bodies move through the water-containing composition and thereby create agitation which distributes the probiotic microorganisms more evenly in the water-containing composition.

The unattached solid bodies preferably have a density which is significantly different from the density of the water-containing composition, i.e. either a significantly higher density or a significantly lower density than the water-containing composition.

The unattached solid bodies may for example have a density of at least 1.5 kg/L. For example, the unattached solid bodies may for example have a density of at least 2 kg/L or even higher densities such as e.g. at least 3 kg/L or at least 5 kg/L.

Alternatively, the unattached solid bodies may have a density of at most 0.9 kg/L. For example, the unattached solid bodies may for example have a density of at most 0.8 kg/L or even higher densities such as e.g. at most 0.8 kg/L or at least 5 kg/L.

At least one of the unattached solid bodies preferably has a volume of least 0.2 cm³. For example, at least one of the unattached solid bodies may have a volume of least 0.5 cm³. Alternatively, at least one of the unattached solid bodies may have a volume of least 1 cm³.

Yet an aspect of the invention pertains to a disposable, sealed first secondary container as defined herein comprising a water-containing composition.

The kit is preferably used by opening the primary container and then opening the secondary container(s). Optionally, the support agent may be transformed into a size which fits the wound to be treated, e.g. by cutting the support agent. The support agent may then be applied to the wound and the water-containing composition of the first secondary container is applied to the support agent.

Alternatively, the support agent is transformed into the desired size, e.g. by cutting, the water-containing composition is applied to the support agent and the wetted support agent is then applied to the wound.

Alternatively, the water-containing composition may be applied to the support agent before optional cutting and before the support agent is applied to the wound.

Once the support agent has been applied to the wound the support agent is fixed by means of one or more fixation member(s) as defined herein.

It is preferred that the first secondary container, before opening, is shaken by hand or turned upside down one or more times to obtain a more uniform distribution of the probiotic microorganisms.

In some preferred embodiments of the invention, the probiotic composition comprises sufficient water-containing composition to allow for rinsing the wound in water-containing composition before the combination of support agent and water-containing composition is applied to the wound. The initial rinsing step has the effect of removing at least some of the colonizing microorganisms of the wound and is believed to accelerate the onset of the beneficial effects of the probiotic composition.

In some embodiments of the invention, the kit contains a third secondary container which contains extra water-containing composition for rinsing the wound prior to the application of the support agent and the water-containing composition of the first secondary container. The third secondary container is preferably designed and produced using one or more embodiments described in the context of first second container. It should be noted that the third secondary container may contain more or less water-containing composition than then first secondary container. It should furthermore be noted that the first and third secondary container of a specific kit need not be of the same design as long as both containers provide for effective storage of the water-containing composition.

The third secondary container typically contains an amount of water-containing composition in the range of 5-200 mL, such as e.g. in the range of 10-100 mL.

The invention provides a method of producing a packaged probiotic composition, comprising the steps of:

a) providing a water-containing composition containing a viable first probiotic microorganism,
b) providing a support agent as defined herein,
c) optionally, contacting the support agent with the water-containing composition,
d) packaging the combination of the support agent and the water-containing composition in a suitable container.

The packaged probiotic composition is preferably manufactured under aseptic conditions, and may for example employ a class 100,000 clean room.

Furthermore, the packaging of step d) is performed under conditions that minimize loss of viability of the probiotic microorganism(s). Thermal processes such as welding should be applied carefully to avoid damaging the probiotic microorganisms.

Yet an aspect of the present invention pertains to the packaged probiotic composition of the invention for prophylactic or therapeutic use for an animal or human. In particular, the probiotic composition is for use as a wound dressing for treatment of wounds or tissues and for treatment and prevention of colonization or infection by pathogenic microorganisms at the wound or tissue site. The probiotic composition may also be for use for re-establishing the bacterial balance of the wound.

A further aspect of the present invention pertains to a method of treating an animal or human subject having a colonized wound or tissue, or being at risk of having a colonized wound or tissue, the method comprising the steps of:

1) providing a packaged probiotic composition;
2) opening the container in which the probiotic composition has been packaged, and
3) applying the probiotic composition to the colonized wound or tissue.

The probiotic composition should preferably be applied so that the first probiotic microorganism is at least in liquid communication with the wound or tissue to be treated. It may furthermore be preferred that the first probiotic microorganism contacts the wound or tissue to be treated.

An advantage of the present packaged probiotic composition is that is reduces or prevents the bad smell from colonized wounds.

The present invention has been described above with reference to specific embodiments. However, other embodiments than the above described are equally possible within the scope of the invention. The different features and steps of various embodiments and aspects of the invention may be combined in other ways than those described herein unless it is stated otherwise.

### EXAMPLES

### Example 1a

### Preparation of a Water-Containing Composition

A water-containing composition was prepared by mixing 800 grams of sugar from Danisco, 16000 grams of 35 degrees C. hot water and a probiotic blend of 1 gram Lactobacillus Acidophilus, 1 gram of Bifidobacterium Animalis and 1 gram of Lactobacillus Casai from Chr. Hansen.

The composition was allowed to ferment for 3 weeks until all sugar had been metabolised by the probiotic blend. The pH of the resulting water-containing composition was pH 4.

### Example 1b

### A Water-Containing Composition

1 liter of “Vita Biosa Probiotic urter” having a pH of 3.4 was purchased from Biosa Danmark and used as an alternative water-containing composition.

### Example 2

### Preparation of Packaged Wound Dressings

A PVA foam from Mondomed (8×12×0.6 cm) was impregnated with the water-containing composition from example 1b by dipping the foam into the water-containing composition. After 1 minute, the foam was removed and excess liquid was allowed to drip off. The sample was called 2a.

A non-woven fabric comprising super absorbent fibres (SAF), Oasis Type 2577 from Technical Absorbent Inc., (comprising 75% SAF of polyacrylate and 25% polyester fibres) was impregnated with the water-containing composition by dipping the fabric into the water-containing composition 1b. After 1 minute, the fabric was removed and excess liquid was allowed to drip of. The sample was called 2b.

An Alginate Wound dressing from Kanglidi Medical (a non-woven fabric) was impregnated with the water-containing composition by dipping the non-woven fabric into the water-containing composition 1b. After 1 minute, the non-woven fabric was removed and excess liquid was allowed to drip of. The sample was called 2c.

A Chitosan Wound dressing from Kanglidi Medical (a non-woven fabric) was impregnated with the water-containing composition by dipping the non-woven fabric into the water-containing composition 1b. After 1 minute, the non-woven fabric was removed and excess liquid was allowed to drip of. The sample was called 2d.

All samples were subsequently packed and sealed in zipper pouches and subjected to the testing described in Example 4.

### Example 3

### Preparation of Packaged Amorphous Hydrogels

20 grams of superabsorbent polyacrylate particles from BASF was blended with 500 grams of probiotic composition and 500 grams of saline water (0.9% NaCl). The amorphous hydrogel was packed in a syringe and called 3a.

35 grams of Chitosan Primex (Chitoclear high molecular weight) was blended with 500 grams of probiotic composition and 500 grams of saline water (0.9% NaCl). The amorphous hydrogel was packed in a syringe and called 3b.

The two samples were subsequently packed in syringes and subjected to the testing described in Example 5.

### Example 4

### Comparative Testing—Wound Dressings

The wound dressings from example 2 were tested in relation to stability and how the water containing composition influenced the stability and appearance of the support agent. The samples were stored at 35 degree Celsius. After 48 hours, the samples were visually inspected in relation to coloration, formation of gas in the pouch, visual evaluation of degradation of the support agent and formation of gas bobbles in the wound dressing. After 5 days, the viability of the probiotic bacteria was analysed. The results are summarized in Table 1.

TABLE 1
Comparison of wound dressings

| | Visual inspection | | | |
|---|---|---|---|---|
| Sample | Color | Gas formation | Other comments | Conclusion |
| 2a | Off | No | Nice even | The support agent is not |
| | White to | | appearance, same | affected by the water- |
| | light | | color as before | containing composition, |
| | brown | | heating. | but reduces the viability of |
| | | | | the probiotic bacteria |
| | | | | significantly. |
| 2b | Light | No | Nice even | The support agent is not |
| | brown | | appearance, same | affected by the water- |
| | | | color as before | containing composition, |
| | | | heating. | and is concluded to be |
| | | | | useful as support agent. |
| 2c | Brown | No | Slightly more dark | The support agent is |
| | | | color after testing, | affected by the water- |
| | | | but with an even | containing composition to |
| | | | color distribution | such a degree that it is not |
| | | | on the sample. Has | useful as a support agent. |
| | | | loss some liquid. | |
| 2d | Dark | Yes, but | Partly soluble, | The support agent is |
| | brown | minimal | partly gelled (soft | affected by the water- |
| | | | gel). Uneven color | containing composition to |
| | | | distribution on the | such a degree that it is not |
| | | | sample. | useful as a support agent. |

Based on the above test, it is concluded that sample 2b is a stable and useful as a support agent. Samples 2c and 2d are significantly affected by the water-containing composition and are therefore unsuitable as support agents in probiotic compositions where the water-containing composition contacts the support agent during storage of the probiotic composition. The support agent of sample 2a reduced significantly the probiotic bacteria and is therefore not found biocompatible and suitable for the present invention.

### Example 5

### Comparative Testing—Amorphous Hydrogels

The samples were stored at 35 degree Celcius for 48 hours. After the 48 hours, the samples were visually inspected in relation to coloration, formation of gas in the syringes, visual evaluation of degradation of the support agent and formation of gas bubbles in the hydrogel. The results are summarized in Table 2.

TABLE 2
Comparison of hydrogels

| | Visual inspection | | | |
|---|---|---|---|---|
| Sample | Color | Gas formation | Other comments | Conclusion |
| 3a | Light | No | The sample has a | The support agent is not |
| | brown | | nice even | affected by the water |
| | | | appearance, same | containing composition, |
| | | | color as beforeheating. | and is concluded to be |
| | | | Seems to maintain | useful as support agent. |
| | | | its original viscosity. | |
| 3b | Dark | No | More dark color after | The support agent is |
| | brown | | testing, the sample | affected by the water |
| | | | seems to have lost | containing composition |
| | | | some viscosity. | to such a degree, that |
| | | | | the sample is not useful |
| | | | | as support agent. |

Sample 3a is evaluated to be useful as a support agent in the present invention, while sample 3b is found not useful as it is affected significantly by the water-containing composition and shows clear signs of degradation. 3b is therefore unsuitable as support agent in probiotic compositions where the water-containing composition contacts the support agent during storage of the probiotic composition.

### Example 6

### Production and Use of a Kit

An example of the production and use of a kit under the present invention is provided in this example.
1) Pads of Oasis fibres fabric (Type 2577) of 0.2×5×5 cm³, 0.2×10×10 cm³, 0.2×10×20 cm³ or 0.2×20×20 cm³ are packed individually in containers comprising a tray at the bottom, in which the pad is placed. The container is then sealed and sterilized.
2) The water-containing composition is produced as described in Example 1A, filled into containers under aseptic conditions and sealed.
3) The container holding the fibre fabric pad and the container holding the water-containing composition are then packaged in a suitable box as a kit.

The water-containing composition is manually applied evenly on the fibre fabric pad. The amount applied depends on the exudate level (none/low/medium/high) of the wound. A pad applied on a highly exudating wound could be partially saturated (i.e. 15.0-30.0 ml at a 0.2×10×10 cm³ fibre fabric), whereas a nearly saturated fibre fabric pad (i.e. 30.0-37.5 ml at a 0.2×10×10 cm³ fiber pad) could be applied to a low exudating wound.

If desired, the fibre fabric pad can be cut to fit the shape of the wound. The fibre fabric pad including water-containing composition is preferably changed every day or every second day depending on the characteristics of the wound.

1. A packaged probiotic composition, the probiotic composition comprising:
a water-containing composition containing a viable first probiotic microorganism, said water-containing composition contains water in an amount of at least 50% (w/w) relative to the total weight of the water-containing composition, and
a support agent.

2. The packaged probiotic composition according to claim 1, which is a packaged kit containing
a sealed first secondary container comprising the water-containing composition, and
the support agent.

3. The packaged probiotic composition according to claim 2, wherein amount of water-containing composition in the sealed first secondary container is in the range of 50-200% of the water holding capacity of the support agent.

4. The packaged probiotic composition according to claim 2, wherein the support agent capable of covering a skin area in the range of 1-500 cm².

5. The packaged probiotic composition according to claim 2, wherein the sealed first secondary container contains a mechanism for agitating the water-containing composition before use.

6. The packaged probiotic composition according to claim 1, wherein the water-containing composition has a pH in the range of pH 2-6.

7. The packaged probiotic composition according to claim 1, wherein the support agent is water-insoluble.

8. The packaged probiotic composition according to claim 1, wherein the first probiotic microorganism is a bacterium or a fungus.

9. The packaged probiotic composition according to claim 1, wherein the probiotic composition comprises at least 0.1 mL water-containing composition.

10. The packaged probiotic composition according to claim 1, wherein the weight ratio between the support agent and the water-containing composition is at most 1:1.

11. The packaged probiotic composition according to claim 1, wherein the total amount of carbon-containing nutrients having a molecular weight of at most 5000 g/mol in the water-containing composition is at most 0.5% (w/w) relative to the weight of the water-containing composition.

12. The probiotic composition according to claim 1, wherein the support agent comprises one or more material(s) in a form selected from the group consisting fibres, a foam, a gel network, a gel-forming agent, and a combination thereof.

13. The packaged probiotic composition according to claim 1, wherein the support agent is a water-insoluble gel forming agent.

14. The packaged probiotic composition according to claim 1, wherein the water-containing composition contacts the support agent.

15. The packaged probiotic composition according to claim 1, wherein the water-containing composition does not contact the support agent while the probiotic composition is in a first state, but wherein the probiotic composition can be transformed into a second state where the water-containing composition contacts the support agent.

16. The packaged probiotic composition according to claim 1, the packaged probiotic composition having a shelf-life of at least 3 months when kept at a temperature of 23 degrees C. and a relative humidity of 50%.

17. A method of producing a packaged probiotic composition, the method comprising the steps of:
a) providing a water-containing composition containing a viable first probiotic microorganism,
b) providing a support agent,
c) optionally, contacting the support agent with the water-containing composition, and
d) packaging the combination of the support agent and the water-containing composition in a suitable primary container.

18. A method of treating a human or animal subject having a colonized wound or tissue, or being at risk of having a colonized wound or tissue, the method comprising the steps of:
1) providing a packaged probiotic composition according to claim 1,
2) opening the primary container in which the probiotic composition has been packaged, and
3) applying the probiotic composition to the colonized wound or tissue.

19. The probiotic composition according to claim 1 for use in treatment or prevention of colonized wound or tissue.
